(19) United States
(12) Patent Application Publication
(10) Pub. No.: US 2010/0243500 A1
(43) Pub. Date: Sep. 30, 2010

(54) MULTILAYER INDIVIDUAL PACKAGE FOR ABSORBENT ARTICLES

(76) Inventors: Kimberly Nichole McConnell, Morrow, OH (US); David Andrew Dalton, Loveland, OH (US); Virginia Anne Lenahan, Cincinnati, OH (US); Jeanne Marie Hughes, Loveland, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/414,885

(22) Filed: Mar. 31, 2009

(51) Int. Cl. A61B 19/00; B65D 85/00; A61F 13/551

(52) U.S. Cl. 206/440; 206/459.5; 604/385.02

(57) ABSTRACT

An individually packaged absorbent article including an absorbent article and a package. The package can enclose the absorbent article and can have an outer surface and an inner surface. In certain embodiments, the package can include a package material having a first component that is a polymeric film, and a second component that is an absorbent material. The first material and the second material can be joined together to form the package material, wherein the package material is oriented such that the first component forms the outer surface of the package and the second component forms the inner surface of the package.

### FIELD OF THE INVENTION

This invention relates to packages for individual absorbent articles, and more particularly, to multilayer individual packages for absorbent articles.

### BACKGROUND OF THE INVENTION

Absorbent articles, such as absorbent articles for feminine hygiene, are generally used to absorb body exudates, such as menstrual or other body fluids. Certain types of absorbent articles, such as, for example, sanitary napkins, tampons, incontinence pads, and similar articles can be individually packaged. These individual packages can protect the article and can facilitate hygiene, ease of use and identification, and ease of carrying.

Currently available individual packages can have one or more drawbacks. For example, current individual packages can be noisy during carrying and use, which can be undesirable for a discreet user experience. In addition, some packages may not adequately conceal the absorbent article. Furthermore, such packages can feel thin or flimsy, and can have reduced durability and/or can provide a perception of a lower quality product.

Typically, individual packages can be formed from a plastic film, which can protect the absorbent article while providing a smooth and/or shiny outer surface, or formed from paper, which can be biodegradable and easy to open. Both plastic film and paper packages, however, can be noisy. In addition, paper packages can tear and/or become soiled during carrying and use. Other individual packages can include an exterior nonwoven layer that may provide a soft package. The nonwoven exterior of such packages, however, may become soiled during carrying and use, and may not provide a desirable smooth and/or shiny appearance.

As such, it would be desirable to provide an individual package for an absorbent article that can have improved quietness and/or softness, and/or an improved luxurious and attractive feeling and appearance.

### SUMMARY OF THE INVENTION

An individually packaged absorbent article including an absorbent article and a package is provided. The package can enclose the absorbent article and can have an outer surface and an inner surface. In certain embodiments, the package can include a package material having a first component that is a polymeric film, and a second component that is an absorbent material. The first component and the second component can be joined together to form the package material, wherein the package material is oriented such that the first component forms the outer surface of the package and the second component forms the inner surface of the package.

Also provided is an individually packaged absorbent article including an absorbent article and a package enclosing the absorbent article. The package can have an outer surface and an inner surface disposed opposite the outer surface, a periphery, a longitudinal axis, a transverse axis, a length measured parallel to the longitudinal axis, a width measured parallel to the transverse axis, a top, a bottom, and two sides. The package can include a package material having a first component that is a polymeric film, and a second component that is an absorbent material. The first component and the second component can be joined together to form the package material, wherein the package material is oriented such that the first component forms the outer surface of the package and the second component forms the inner surface of the package. In certain embodiments, the package material can be formed into a pocket that can be configured to enclose at least a portion of the absorbent article.

Further provided is an individually packaged absorbent article including an absorbent article and a reclosable package enclosing the absorbent article. The package can have an outer surface and an inner surface disposed opposite the outer surface and can include a package material having a first component that is a printed polyolefin film, and a second component that is an absorbent material. The first component and the second component can be joined together to form the package material, and the package material can be oriented such that the first component forms the outer surface of the package and the second component forms the inner surface of the package.

### BRIEF DESCRIPTION OF THE DRAWINGS

FIG. 1 is a plan view of an individual package.

FIG. 2 is a plan view of an individual package.

FIG. 3 is a cross-sectional view of an individual package, taken along line 3-3 of FIG. 2.

FIG. 4 is a cross-sectional view of a package material.

### DETAILED DESCRIPTION OF THE INVENTION

The present invention relates to individual packages for absorbent articles, such as, e.g., absorbent articles for feminine hygiene. The individual packages can include a package material that can provide a liquid impermeable exterior and an absorbent interior. For example, in certain embodiments, the package material can have a first component that is a polymeric film and a second component that is an absorbent material. In addition, the package material can be oriented such that the film component forms the exterior of the package and the absorbent component forms the inside of the package. Surprisingly, the film outer layer and absorbent material inner layer can provide a quieter, softer package that can provide benefits of a plastic wrapper, such as, e.g., a liquid permeable exterior and a smooth and/or glossy surface that can be amenable to high contrast printing. In addition, such package material can provide a thick and/or cushioned feel and/or improved concealment of an absorbent article contained therein. Furthermore, in certain embodiments, the absorbent interior can retain body fluid from a used article and thus can provide for a cleaner disposal experience. In certain embodiments, the individual package can include a closure and/or sealing device for sealing the used article within the package.

As used herein, the term “absorbent” refers to materials that can hold, retain, and/or contain body fluids.

As used herein, the term “absorbent article” refers to devices that absorb and contain body exudates, and more specifically, refers to devices that are placed against or in proximity to the body of the wearer to absorb and contain various exudates discharged from the body. Absorbent articles include, for example, sanitary napkins, incontinence articles, interlabial pads, tampons, and pantyliners.

The term “disposable” is used herein to describe articles that are intended to be discarded after a single use. That is, they are not intended to be laundered or otherwise restored or reused.

As used herein, the term “feminine hygiene articles” refers to articles that typically can be intended for feminine use, such as, e.g., absorbent articles, such as, e.g. sanitary napkins, liners, tampons, interlabial articles, incontinence articles; and pessaries.

As used herein, the term “tampon” refers to any type of absorbent structure such as, e.g., an absorbent mass, that can be inserted into the vaginal canal or other body cavity for the purpose of, such as, e.g., absorbing fluid, aiding in wound healing, and/or for delivering materials, such as moisture or active materials such as medicaments. The term “tampon” can also include the combination of an absorbent structure with any type of applicator that can be associated with the absorbent structure to facilitate insertion of a tampon into the vaginal canal or other body cavity. A tampon can include any known tampon configuration such as, for example, digital tampons, tampons with traditional plunger type applicators, and/or tampons with compact applicators, such as, e.g., tampons described in U.S. Pat. Nos. 4,726,805; 4,846,802; 4,960,417; 5,087,239; 5,279,541; 6,258,075; 6,478,763; or any other tampon.

As used herein, the term “nonwoven” can refer to a web or fabric having a structure of individual fibers or threads which are interlaid, but not in a regular, repeating manner as in a woven or knitted fabric. Nonwoven webs or fabrics can be formed from many processes, such as, for example, meltblowing processes, spunbonding processes, hydroentangling processes, and bonded carded web processes.

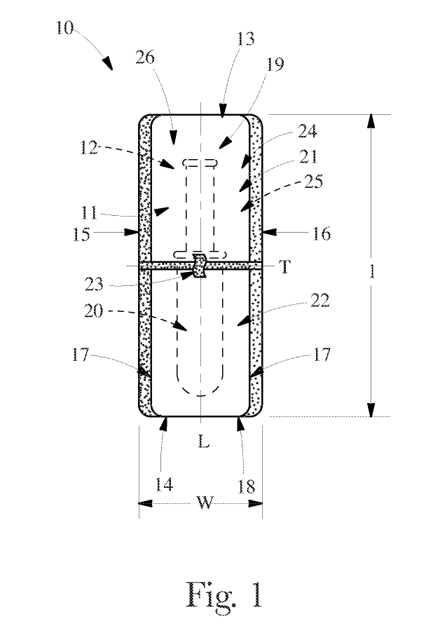

FIG. 1 shows one embodiment of an individual package 10. The package 10 has an outer surface 11, an inner surface 12, a top 13, a bottom 14, a length (l), a width (w), a longitudinal axis (L), and a transverse axis (T). As shown in FIG. 1, the package 10 also has a first side 15 and a second side 16 that can include side seals 17. The package 10 can substantially enclose an absorbent article 20. In addition, the package 10 can be formed of a package material 26 formed from a first component 24, such as, e.g., a polymeric film, and a second component 25, such as, e.g., an absorbent material, such as, e.g., a nonwoven or formed film, or other suitable material. The first component 24 and the second component 25 can be joined together to form the package material 26. As shown in FIG. 1, the package material 26 can be oriented such that the first component 24 forms the outer surface 11 of the package 10 and the second component 25 forms the inner surface 12 of the package 10. In certain embodiments, the package 10 can include a pocket 22 that can be configured to enclose at least a portion of the absorbent article 20. As shown in FIG. 1, the pocket 22 can be formed by folding the package material 26 along fold line 18, which can be substantially parallel to the transverse axis (T). In addition, or alternatively, the package material 26 can be formed into a flap 21 that can be configured to function as a closure for the package 10. In certain embodiments, the flap 21 can be formed by folding the package material 26 along fold line 19, which can be substantially parallel to the transverse axis (T) and/or to fold line 18. In addition, or alternatively, the package, such as, e.g., the flap 21, can include a tab 23, such as, e.g., a resealable tab.

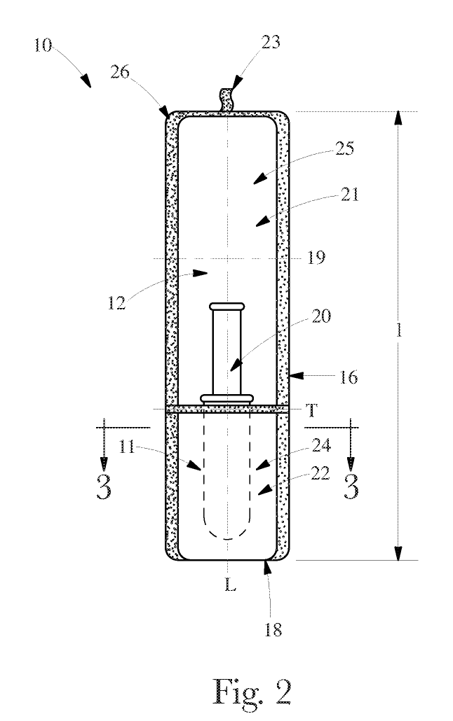

FIG. 2 shows one embodiment of an individual package 10. The package 10 has an outer surface 11 and an inner surface 12, a longitudinal axis (L), and a transverse axis (T). The package 10 can substantially enclose an absorbent article 20. As shown in FIG. 2, the package 10 can be formed of a package material 26 having a first component 24, such as, e.g., a polymeric film, and a second component 25, such as, e.g., an absorbent material, such as, e.g., a nonwoven or formed film, or other suitable material. The first component 24 and the second component 25 can be joined together to form the package material 26. As shown in FIG. 2, the package material 26 can be oriented such that the first component 24 forms the outer surface 11 of the package 10 and the second component 25 forms the inner surface 12 of the package 10. In certain embodiments, the package 10 can include a pocket 22 that can be configured to enclose at least a portion of the absorbent article 20. As shown in FIG. 2, the pocket 22 can be formed by folding the package material 26 along fold line 18, which can be substantially parallel to the transverse axis (T). In addition, or alternatively, the package material 26 can be formed into a flap 21 that can be configured to function as a closure for the package 10. In certain embodiments, the flap 21 can be formed by folding the package material 26 along fold line 19, which can be substantially parallel to the transverse axis (T) and/or to fold line 18. In addition, or alternatively, the package, such as, e.g., the flap 21, can include a tab 23, such as, e.g., a resealable tab.

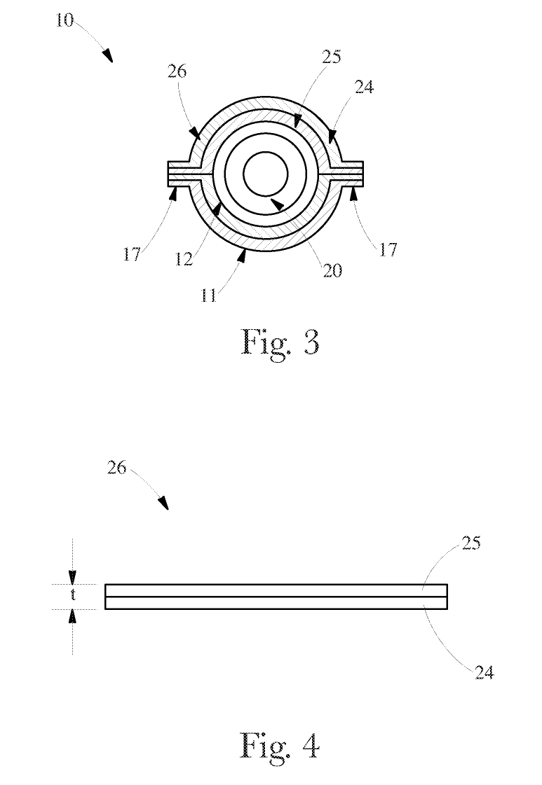

FIG. 3 shows a cross-sectional view of a package 10, such as, for example, a cross-sectional view along line 3-3 of the package 10 shown in FIG. 2. The package 10 can have an outer surface 11 and an inner surface 12. As shown in FIG. 3, the package 10 can be formed of a package material 26 formed from a first component 24, such as, e.g., a polymeric film, and a second component 25, such as, e.g., an absorbent material, such as, e.g., a nonwoven material, a formed film, or other suitable material. The first component 24 and the second component 25 can be joined together to form the package material 26. As shown in FIG. 3, the package material 26 can be oriented such that the first component 24 forms the outer surface 11 of the package 10 and the second component 25 forms the inner surface 12 of the package 10. In certain embodiments, two layers of package material 26 can be joined at side seals 17 to form package 10.

FIG. 4 shows a package material 26 prior to forming package 10. As shown in FIG. 4, package material 26 can be formed from a first component 24, such as, e.g., a polymeric film, and a second component 25, such as, e.g., an absorbent material, such as, e.g., a nonwoven. In certain embodiments, the package material 26 can have a thickness (t).

The package material has a first component and a second component. In certain embodiments, the first component of the package material can be a polymer, such as, e.g., a polymeric film. Suitable polymers include, for example, polyolefins, such as, e.g., polyethylene, polypropylene, polyesters, such as, e.g., synthetic polyesters, polyamides, polyvinyl chlorides, ethylene-vinyl acetate copolymers, and/or other suitable films. In certain embodiments, the first component can be generally liquid repellent. In addition, or alternatively, the first component can be considered non-flushable, non-water-degradable, and/or generally insoluble in water. In certain embodiments, the absorbent material can have a basis weight of less than about 30 gsm, such as, e.g., less than about 25 gsm, less than about 20 gsm, less than about 15 gsm, or less than about 10 gsm, or any other suitable basis weight. In certain embodiments, the first component can be printable, such as, e.g., printed with one or more images, such as, e.g., printed with one or more absorbency indicators, such as, e.g., printed with one or more colors corresponding to absorbency level. In certain embodiments, the first component can be amenable to high definition and/or high contrast printing.

In certain embodiments, the second component of the package material can be an absorbent material, such as, e.g., a nonwoven, a formed film, a paper, or a fabric. In certain embodiments, the second component is a nonwoven, and can include fibers comprised of polymers such as polyethylene, polypropylene, polyester, cellulose, rayon, cotton, super absorbent material such as polyacrylate, or combinations thereof. In addition, the second component can be considered non-flushable, non-water-degradable, and/or generally insoluble in water. Alternatively, in certain embodiments, the second component can be considered degradable, water soluble, and/or flushable, such as, e.g., when the second component comprises certain papers or other degradable material. In certain embodiments, the absorbent material can have a basis weight of less than about 30 gsm, such as, e.g., less than about 25 gsm, less than about 20 gsm, less than about 15 gsm, or less than about 10 gsm, or any other suitable basis weight.

The first component and the second component can be joined in any suitable manner to form the package material, such as, e.g., by adhesive bonding, thermal bonding, ultrasonic bonding, and the like. While complete bonding of the first component and the second component may not be necessary, in certain embodiments, the bonding should be sufficient to facilitate that the components act as a unit, e.g., bending out-of-plane together. In certain embodiments, the first component can have a surface area that is greater in size than the surface area of the second component. For example, when the first component is greater in size than the second component, the first component can be joined to itself, such as, e.g., substantially enclosing the second component, when the package material is formed into the package.

The package material will typically be thick and strong enough to provide the desired soft and thick feel. For example, in certain embodiments, the package material will be greater than about 0.1 mm thick, such as, e.g., greater than about 0.2 mm thick, greater than about 0.3 mm thick, greater than about 0.4 mm thick, greater than about 0.5 mm thick, greater than about 0.6 mm thick, greater than about 0.7 mm thick, greater than about 0.8 mm thick, greater than about 0.8 mm thick, greater than about 0.9 mm thick, greater than about 1 mm thick, greater than about 2 mm thick, greater than about 3 mm thick, greater than about 4 mm thick, greater than about 5 mm thick, or any other suitable thickness. In addition, in certain embodiments, the package material can have a high tear resistance. In certain embodiments, the package material can be considered non-flushable, non-water-degradable, and/or generally insoluble in water.

The package can be constructed in any suitable manner, such as, e.g., constructed of one connected piece of package material or constructed from multiple pieces of material sufficiently joined together such that it substantially acts as one connected piece of package material. In certain embodiments, the package can be formed by closing the package material via heat-sealing onto itself before and/or after wrapping the absorbent article. In addition, or alternatively, the package can be glued, embossed, crimped, sewed, stitched, entangled, mechanically interlocked, cold pressure welded, ultrasonic bonded, and/or otherwise bonded or sealed.

The package can include an opening device, such as, e.g., a flap, a tab, a pull, a notch, or any other suitable opening device. Typically, the individual absorbent article will be sealed within the individual package prior to use and the package will need to be opened by a user to access the absorbent article within. In certain embodiments, the package can include a flap that the user can release to open the package. Alternatively, or in addition, the package can include one or more tabs that the user can pull to open the package. In certain embodiments, the package can include one or more lines of weakness, that can, in certain embodiments, be combined with one or more additional opening devices. For example, in certain embodiments, the package can include a closure such as, e.g., a flap, that can be lifted to expose a line of weakness that can be broken to access the absorbent article. In addition, the flap and/or any other suitable portion of the package can include a sealing device that can allow the flap to be resealed over a used absorbent article or related material.

In certain embodiments, the package can include a line of weakness that can function, for example, to allow access to the absorbent article packaged therein. A line of weakness can be formed in any suitable manner, such as, e.g., mechanically and/or thermally, such as, for example, by using a laser and/or chemically. A line of weakness can include a plurality of weakness points arranged in a row. The row can be straight, bent, angled, curved, and/or can change direction. In certain embodiments, one or more of the individual weakness points can overlap to form the line of weakness. In addition, or alternatively, one or more of the individual weakness points can be spaced apart from one another to form the line of weakness. The one or more lines of weakness can be formed by perforations or by depressions.

In certain embodiments, at least a portion of an absorbent article and/or related material will need to be disposed after use. The user can place the used article into the package for disposal. For example, in certain embodiments, the package can include a pocket and the pocket can act to contain at least a portion of the used absorbent article or portion thereof. In certain embodiments, a portion of the package, such as, e.g. a flap of the package, can be pulled over the used article once it is placed into the package for transportation and/or disposal, such as, e.g. to provide increased discretion and cleanliness during transportation and/or disposal. Alternatively, or in addition, the package can be configured such that it can be wrapped around the used article to facilitate disposal.

In certain embodiments, the package can include a sealing device for sealing a portion of the package over the used article once it is placed into the package for transportation and/or disposal. Any suitable device for sealing the package can be used, such as, e.g., adhesive, glues, tapes, thermal bonds, and/or mechanical fasteners. In certain embodiments, the device can include a portion of the surface of the package can be covered with adhesive, cohesive, or other joining means. In addition, or alternatively, the device can include a sealing tab that can have, for example, a pressure sensitive adhesive or other suitable closure means disposed on one or more surfaces of the sealing tab.

The package can be formed in any suitable configuration. Suitable configurations can include, for example, a tampon package, a sanitary napkin release paper wrapper (tri-fold) or bi-fold, an incontinence pad wrapper, a liner package, or any other suitable configurations.

In certain embodiments, the feminine device can be a tampon. The tampon can include a pledget that can include one or more absorbent materials. The materials for the tampon can be formed into a fabric, web, or batt that is suitable for use in the tampon by any suitable process such as, for example, airlaying, carding, wetlaying, hydroentangling, or other known techniques.

The pledget can be constructed from a wide variety of liquid-absorbing materials commonly used in absorbent articles. Such materials include, for example, rayon (such as GALAXY rayon (a tri-lobed rayon) or DANUFIL rayon (a round rayon), both available from Kelheim Fibres GmbH of Kelheim, Germany), cotton, folded tissues, woven materials, nonwoven materials, synthetic and/or natural fibers or sheeting, comminuted wood pulp, which is generally referred to as airfelt, foams, or combinations of these materials. Examples of other suitable materials include: creped cellulose wadding; meltblown polymers including coform; chemically stiffened, modified or cross-linked cellulosic fibers; synthetic fibers such as crimped polyester fibers; peat moss; foam; tissue including tissue wraps and tissue laminates; or any equivalent material or combinations of materials, or mixtures of these. Additionally, superabsorbent materials, such as superabsorbent polymers or absorbent gelling materials can be incorporated into the tampon.

The pledget can have any suitable shape, size, material, or construction prior to compression and/or shaping. For example, the pledget can include a rolled, tubed, or flat construction of an absorbent that can be a circle, an oval, a semi-circle, a triangle, a chevron shape, an H shape, a bow-tie shape, or any other suitable shape, such as, e.g., shapes described in, for example, U.S. Pat. Nos. 3,738,364; 5,911,712; 6,740,070; 6,887,266; and 6,953,456.

In certain embodiments, all or a portion of the tampon can be compressed into a substantially cylindrical configuration, however, other shapes are possible. These can include shapes having a cross section or cross-section element that can be described as rectangular, triangular, trapezoidal, semi-circular, hourglass, or other suitable shapes. In certain embodiments, the tampon can have a radially compressed rolled construction. The tampon can be constructed by rolling and radially compressing a pledget. In addition, or alternatively, the tampon can include an asymmetric insertion end, such as, e.g., tampons disclosed in U.S. patent application Ser. Nos. 11/526,041 and 11/525,513.

A tampon can comprise one or more overwraps, such as, e.g., one or more overwraps attached to the compressed tampon. The overwrap can be any suitable material, such as, for example, rayon, cotton, bicomponent fibers, polyethylene, polypropylene, other suitable natural or synthetic fibers known in the art, and mixtures thereof. In certain embodiments, the tampon can comprise an overwrap material that substantially encloses the compressed tampon. In certain embodiments, the overwrap can extend from the withdrawal end of the tampon.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An individually packaged absorbent article comprising:
an absorbent article;
a package enclosing the absorbent article, the package having an outer surface and an inner surface disposed opposite the outer surface, the package comprising a package material having a first component that is a polymeric film, and a second component that is an absorbent material, the first component and the second component being joined together to form the package material, wherein the package material is oriented such that the first component forms the outer surface of the package and the second component forms the inner surface of the package.

2. The individually packaged absorbent article of claim 1, wherein the first component is a polyolefin.

3. The individually packaged absorbent article of claim 1, wherein the second component is a nonwoven or a formed film.

4. The individually packaged absorbent article of claim 1, wherein the package material has a thickness greater than about 0.1 mm.

5. The individually packaged absorbent article of claim 1, wherein the second component has a basis weight less than about 30 gsm.

6. The individually packaged absorbent article of claim 3, wherein the package material is water insoluble.

7. The individually packaged absorbent article of claim 1, wherein the package is reclosable.

8. The individually packaged absorbent article of claim 1, wherein the absorbent article is a tampon, a sanitary napkin, an interlabial pad, or an incontinence pad.

9. An individually packaged absorbent article comprising:
an absorbent article;
a package enclosing the absorbent article, the package having an outer surface and an inner surface disposed opposite the outer surface, a periphery, a longitudinal axis, a transverse axis, a length measured parallel to the longitudinal axis, a width measured parallel to the transverse axis, a top, a bottom, and two sides;
the package comprising a package material having a first component that is a polymeric film, and a second component that is an absorbent material, the first component and the second component being joined together to form the package material, wherein the package material is oriented such that the first component forms the outer surface of the package and the second component forms the inner surface of the package; and
wherein the package material is formed into a pocket, the pocket being configured to enclose at least a portion of the absorbent article.

10. The individually packaged absorbent article of claim 9, wherein package includes a flap adapted to enclose the at least a portion of the absorbent article within the pocket.

11. The individually packaged absorbent article of claim 9, wherein the package includes a sealing device adapted to close the pocket.

12. The individually packaged absorbent article of claim 9, wherein the absorbent article is a tampon, a sanitary napkin, an interlabial pad, or an incontinence pad.

13. An individually packaged absorbent article comprising:
an absorbent article;
a reclosable package enclosing the absorbent article, the package having an outer surface and an inner surface disposed opposite the outer surface, the package comprising a package material having a first component that is a printed polyolefin film, and a second component that is an absorbent material, the first component and the second component being joined together to form the package material, wherein the package material is oriented such that the first component forms the outer surface of the package and the second component forms the inner surface of the package.

14. The individually packaged absorbent article of claim 13, wherein the printed polyolefin film includes a printed absorbency identifier.

15. The individually packaged absorbent article of claim 13, wherein the second component is a nonwoven or a formed film.

16. The individually packaged absorbent article of claim 13, wherein the package material has a thickness greater than about 0.1 mm.

17. The individually packaged absorbent article of claim 13, wherein the second component has a basis weight less than about 30 gsm.

18. The individually packaged absorbent article of claim 13, wherein the package material is water insoluble.

19. The individually packaged absorbent article of claim 13, wherein the package includes a resealable tab.

20. The individually packaged absorbent article of claim 13, wherein the absorbent article is a tampon, a sanitary napkin, an interlabial pad, or an incontinence pad.
